(19) United States
(12) Patent Application Publication
(10) Pub. No.: US 2026/0248559 A1
(43) Pub. Date: Aug. 27, 2026

(54) APPARATUS AND METHOD FOR POSITIONING A TUBE

(71) Applicant: Intelligent Ultrasound Limited, Cardiff (GB)

(72) Inventors: Nicholas SLEEP, Cardiff (GB); Stephen MARGETTS, Cardiff (GB); James BOWNESS, Cardiff (GB)

(73) Assignee: Intelligent Ultrasound Limited, Cardiff (GB)

(21) Appl. No.: 18/577,476

(22) PCT Filed: Jun. 28, 2022
(86) PCT No.: PCT/GB2022/051649

§ 371 Date: Jan. 8, 2024

(30) Foreign Application Priority Data
Jul. 9, 2021 (GB) 2109950.2

(51) Int. Cl. A61B 34/20; A61B 90/00; A61J 15/00; A61M 25/01; A61M 25/09

(52) U.S. Cl. CPC A61B 34/20; A61B 90/37; A61B 90/39; A61J 15/0007; A61M 25/0108; A61M 25/09; A61B 2034/2063; A61B 2090/378; A61B 2090/3929; A61M 2210/1053

(57) ABSTRACT

An intracorporal tube for positioning within a patient's body is provided, the tube comprises at least one location marker on, in, or within the tube, the presence and/or position of which marker is detectable by an ultrasound imaging system. In one embodiment, a hollow nasogastric tube, is for insertion into a patient's body. Also depicted is an axial cross-section of the tube, which has a tube wall in which are embedded ultrasonic emitters as active location markers. The emitters are connected by an electrically conductive drive wire.

The present invention relates to an apparatus and method for positioning an intracorporal tube within a body.

### BACKGROUND

Some medical procedures on the body require the positioning of an intracorporal tube, for example, a feeding tube, a draining tube, or a catheter. Often these procedures are of the invasive, blind-ended type wherein the tube is not observable during and/or after insertion. Incorrect placement of an intracorporal tube can be dangerous, even fatal. FIG. 1 shows a known intracorporal tube procedure in the form of a nasogastric tube feeding procedure. In such a procedure, a nasogastric tube 5 is inserted through the nasal (or oral) cavity 3, down the oesophagus 4 and into the stomach 2 of a patient 1. In a nasogastric tube feeding procedure, if the tube is inserted into the lungs by mistake, the patient can come to serious harm, or die, because of introducing substance into the lungs through the tube.

One previously considered approach to this problem has been to use X-rays to confirm the position of the tube after insertion into a body. However, there are several drawbacks to this technique. For one, it may involve moving the patient to a radiology department within the hospital, or else bringing a portable X-ray machine to the patient. Both cases lead to a significant delay as well as the increased costs of providing nurse, porter, and radiographer time. X-rays are a form of ionising radiation, so every X-ray image exposes the patient (and potentially surrounding patients, in the context of a portable X-ray) to a small dose of harmful radiation.

Other non-imaging methods exist to check that the tube is in the stomach, for example pH assessment of fluid aspirated after placement. Although commonly performed, assessment of the gastric pH level is not fool proof, not always feasible, and may be confounded by the presence of feed or other medications in the stomach. Therefore, alternative methods for confirmation of tube placement are needed.

Ultrasound imaging is commonly used to support invasive medical procedures, such as the insertion of needles for ultrasound guided biopsy of tumours, insertion of central venous catheters, or injection of local anaesthetic in ultrasound-guided regional anaesthesia. It has also been suggested that ultrasound could be used to help confirm the placement of intracorporal tubes such as drains and feeding tubes.

In general, it is often difficult to reliably determine the location of tools such as nasogastric tubes on the ultrasound image because such tools have poor echogenicity. In addition, less-experienced practitioners may have difficulty interpreting anatomy on the ultrasound image. This means that they may not reliably be able to decide whether a tool lies within a given anatomical region of interest, even if they can locate the tool on the image. Ultrasound imaging is particularly difficult for the placement of intracorporal tubes, as the tube often has poor visibility on ultrasound and may be obscured by gases (which are poor transmitters of ultrasound).

Embodiments of the present invention aim to address these issues by providing an apparatus for confirming the position of an intracorporal tubing within a body, in particular, an apparatus which can be used with relatively little experience or training.

The present invention is defined in the attached independent claims, to which reference should now be made. Further, preferred features may be found in the sub-claims appended thereto.

### SUMMARY OF THE INVENTION

According to one aspect of the present invention, there is provided an intracorporal tube for positioning within a patient's body, the tube comprising at least one location marker on, in or within the tube, the presence and/or position of which marker is detectable by an ultrasound imaging system.

The tube may have a plurality of location markers at different locations on, or within, the tube. The tube may have one or more active location markers and/or one or more passive location markers.

With multiple location markers, the location of different parts of the tube within the body can be determined. The orientation of the tube may also be established, for example by using multiple location markers. This can be useful, for example, to determine that the tube has coiled or snagged within the body.

The at least one location marker may be an active marker and may comprise an image signal source.

The at least one location marker may be provided in or on a wall portion of the tube. Alternatively, or in addition, at least one location marker may be provided in or on a guide wire of the tube.

Where there is a plurality of location markers, at least two may be configured to produce a discriminable imaging signal in an imaging system such that the positions of the location markers are distinguishable. This may allow identification of parts of the tube the patient's body and their respective locations.

A location marker may be arranged in use to interact with an ultrasound probe, such that the probe causes the marker to emit a detectable response signal.

At least one location marker may be arranged in use to generate, reflect, or modify an ultrasound wave as the imaging signal. An ultrasound signal is beneficial over other types of imaging signal, for example X-rays, since the procedure is fast, inexpensive, non-invasive, and non-ionising, and can be performed by staff who don't hold ionising radiation competencies (completed relevant IRMER training or equivalent).

At least one location marker may be a passive marker and may comprise a region of high echogenicity. The region of high echogenicity may reflect ultrasound more strongly than the surrounding materials and, thus, the location marker will be identifiable, optionally highlighted, in a received ultrasound image.

One or more of the location markers may comprise both an active marker and a passive marker.

One or more of the active location markers may be arranged in use to emit an ultrasound signal which can travel through the body to an external detector or imaging system.

The active markers may be configured to vibrate at a frequency detectable by an ultrasound machine, and/or to pulse to aid visualisation on the ultrasound machine.

Where there is a plurality of active markers, they may be arranged in use to produce distinguishable ultrasound signals. This allows that the location markers are individually identifiable so that the positions of plural parts of the tube may be determined.

The active markers may be arranged in use to vibrate at different frequencies to produce distinguishable ultrasound signals.

The active markers may be configured to pulse or vibrate in an identifiable or distinguishable pattern which may be used to distinguish between them.

The active marker may be a piezo-electric emitter connected to a driver. Such emitters can be manufactured relatively inexpensively and to have small, for example even submillimetre, dimensions, making them ideal for use within a typical intracorporal tube having a diameter of around 4 mm to 20 mm, for example.

In a preferred arrangement, there is a plurality of location markers, with one or more being located at, or towards an end/tip of the tube, preferably a leading end, and/or preferably one or more further markers at one or more locations spaced from the said end, for example at one or more known distances from the end. The plurality of location markers may comprise one marker at a tip of the tube, two markers around 5 cm from the tip and three markers around 10 cm from the tip of the tube. This arrangement may provide sufficient coverage along the length of the tube such that the tube is highlighted along its length on a received ultrasound image.

The tube may be a gastric tube, for example a tube use in a nasogastric or orogastric tube feeding or aspiration procedure. The at least one location marker may be configured to generate, reflect, or modify an ultrasound wave to provide an imaging signal.

The at least one location marker may comprise an etched portion of the tube, such as a laser-etched portion. The etched portion may interact with an incident ultrasound imaging signal such that a property of the signal is modified, which modification may be detected.

According to another aspect of the present invention, there is provided an apparatus for improved detection and/or positioning of an intracorporal tubing within a body, the apparatus comprising: an intracorporal tube; an ultrasound imaging signal detector; and an ultrasound imaging signal processing module.

The apparatus may include an ultrasound imaging signal source.

The intracorporal tube may be according to any statement herein.

The apparatus may further comprise a display for displaying an image received from the imaging signal processing module. This allows a user to view the image.

The imaging signal source and the imaging signal detector may comprise an ultrasound probe.

Alternatively, or in addition, the imaging signal source may comprise the at least one location marker.

According to another aspect of the present invention, there is provided a method for improved detection and/or positioning of an intracorporal tube in a patient's body, the method comprising:
inserting the intracorporal tube into the patient's body, the intracorporal tube comprising at least one location marker; detecting an ultrasound imaging signal from the at least one location marker; and using said ultrasound imaging signal to determine the presence of the or each location marker within the patient's body and/or their spatial relationship.

The method may further comprise processing information representing a position of the or each location marker tube within the intracorporal area of interest to produce a real-time image of the tube and/or its location.

The method may further comprise displaying the real-time image of the tube within the intracorporal area of interest on a display/monitor. A person carrying out an intracorporal procedure can view the real-time image of the display and use it to aid them in carrying out the intracorporal procedure.

The method may further comprise extracting spatial information from the detected imaging signal and applying a deep learning model to the extracted information. The deep learning model may apply standard semantic segmentation techniques to produce an artificially enhanced image of the intracorporal area of interest, which may provide an accurate and detailed picture of an area of a patient's body. Deep learning models may further be used to identify or locate the or each location marker on the ultrasound image. This may allow the user to confirm that the intracorporal tube is placed into the correct anatomical area. This may also allow the system to determine which location markers are within the intended area, and which are definitively outside it.

The method may further comprise overlaying the enhanced image of the intracorporal area of interest and the real-time image of the tube within the intracorporal area of interest.

When the, or each, location marker comprises an active marker the method may comprise using Doppler imaging to interpret imaging signals received from the location marker for improved detection and/or positioning of the tube within the intracorporal area of interest. Doppler imaging can use phase-shift or wavelength-shift information to determine the location of one or more location markers more accurately and, thus, determine the position of the tube.

When the location markers are located on a guidewire of the tube, the method may comprise removing the guidewire from the body after correct positioning of the tube. It may be necessary to remove the guidewire prior to delivering substances to an organ in the body.

The method may further comprise, wherein the location markers are active markers, vibrating or pulsing the markers.

Where there is a plurality of location markers the method may comprise causing the location markers to vibrate at different frequencies and/or to pulse the signals generated by the location markers at different frequencies or in different patterns, to differentiate between the plurality of location markers. The use of differentiable location markers allows the tube to be located accurately within an area of interest in the body.

The method may further comprise obtaining one or more ultrasound images of the intracorporal area of interest from at least two planes, for example at right angles, to provide real-time assessment of the intracorporal area of interest.

The method may further comprise taking three or more ultrasound views of the intracorporal area of interest at different angles to provide an improved real-time assessment of the intracorporal area of interest.

The invention may include any combination of the features or limitations referred to herein, except such a combination of features as are mutually exclusive, or mutually inconsistent.

### BRIEF DESCRIPTION OF THE DRAWINGS

Preferred embodiments of the present invention will now be described, by way of example only, with reference to the accompanying diagrammatic drawings, in which:

FIG. 1 is a schematic diagram of a known nasogastric tube feeding procedure;

FIG. 2 is a schematic sectional view of a tube according to a first embodiment of the present invention;

FIG. 3 is a schematic sectional view of a tube according to a second embodiment of the present invention;

FIG. 4 is a schematic sectional view of a tube according to a third embodiment of the present invention;

FIG. 5 is a schematic sectional view of a tube according to a fourth embodiment of the present invention;

FIG. 6 is a schematic diagram of a nasogastric tube feeding procedure using a tube according to the first, second, third or fourth embodiment;

FIG. 6a is a schematic diagram of an ultrasound image of a nasogastric tube feeding procedure using a tube according to the first or second embodiments;

FIG. 7 shows schematically an apparatus for improved detection/positioning of an intracorporal tubing using a tube according to any of the first, second, third or fourth embodiments of the present invention;

FIG. 8 is a flow chart depicting a method for improved detection/positioning of an intracorporal tube, in accordance with an embodiment of the present invention; and

FIG. 9 is a flow chart depicting a method for producing an artificially enhanced image of an intracorporal area of interest wherein an intracorporal tube is to be positioned.

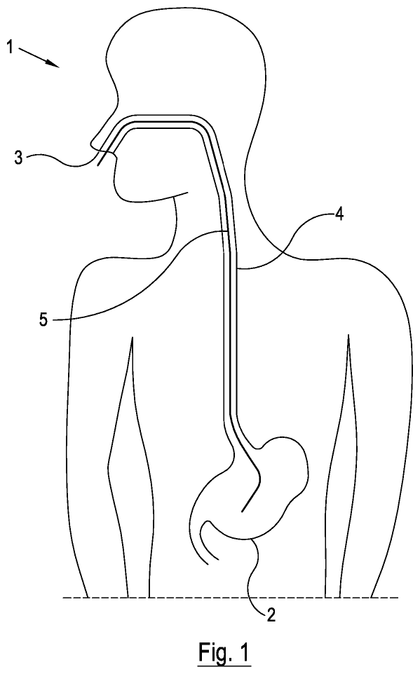

### DETAILED DESCRIPTION

In the embodiments described below, the tube is a nasogastric tube and the signal with which it interacts is an ultrasound signal.

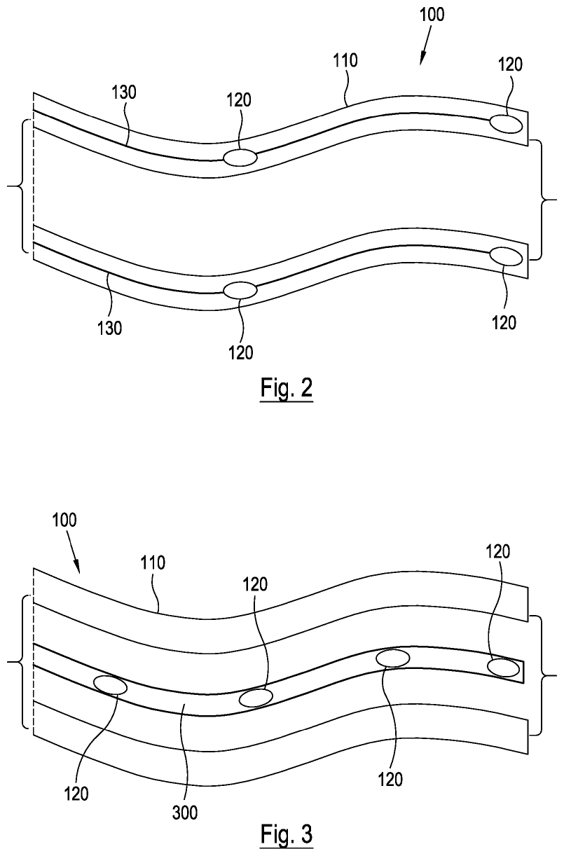

FIG. 2 shows generally at 100 a hollow nasogastric tube for insertion into a patient's body (not shown). FIG. 2 depicts an axial cross-section of the tube 100, which has a tube wall 110 in which are embedded ultrasonic emitters 120 as active location markers. The emitters 120 are connected by an electrically conductive drive wire 130.

FIG. 3 shows an alternative embodiment, in which the tube 100 has a central guide wire 300 in which the active markers 120 are embedded.

Using piezo-electric emitters as active location markers has the benefit that they can be made inexpensively and to a submillimetre size. The piezo-electric emitters 120 are configured to emit an ultrasound signal that can be detected by an ultrasound probe (not shown) located outside the patient's body, as will be described below. In use, the emitters 120 are configured to vibrate at a frequency detectable by the ultrasound machine (e.g., in the range 3-20 MHz). The markers 120 may also be configured to pulse or vibrate with different patterns to aid identification. In one example of the invention, different markers 120 are configured to vibrate at different frequencies and/or to pulse at different frequencies. This difference in vibration and/or pulse frequency allows separate detection of each marker. Thus, various locations on the tube 100 can be identified.

Since the location markers 120 of the embodiment shown in FIG. 2 remain on the tube 100 after its insertion 100 into the intracorporal area interest, follow-on detection and confirmation of placement is possible for the duration that the tube 100 is inside the body. This may be desirable to ensure on-going correct placement of the tube 100 during a procedure, for example to ensure that the tube 100 does not move out of place, or to relocate the tube 100 during the procedure if required.

In the embodiment of FIG. 3, the guidewire 300 comprising the location markers 120 is removable after correct placement of the tube 100. In some examples of the present invention a guidewire comprising location markers is used in combination with a tubing comprising location markers embedded within the tube wall. This provides a tube with a greater number of location markers for improved position monitoring of the tube. It also provides a device the location of which can be continually monitored during a procedure even after removal of the guidewire.

In use, the markers 120 are electrically connected via the drive wire 130, in the case of the FIG. 2 embodiment, to an actuator (not shown). In the example of FIG. 3, the markers 120 are electrically connected to the actuator (not shown) via the guidewire itself.

The number and placement of the location markers 120 in the examples shown in FIGS. 2 and 3 is illustrative. A simple case (not shown) has only a single location marker, optimally positioned at a leading end/tip of the tube. However, there may be any number of location markers. For example, the arrangement of location markers 120 on a tube 100 may be: a single marker at the tip of the tube in the direction of insertion into the intracorporal area, two further markers positioned at around 5 cm from the tip, and three further markers at around 10 cm from the tip. Each of the markers 120 may be configured to vibrate at different frequencies and/or to pulse at different frequencies to be distinguishable from each other.

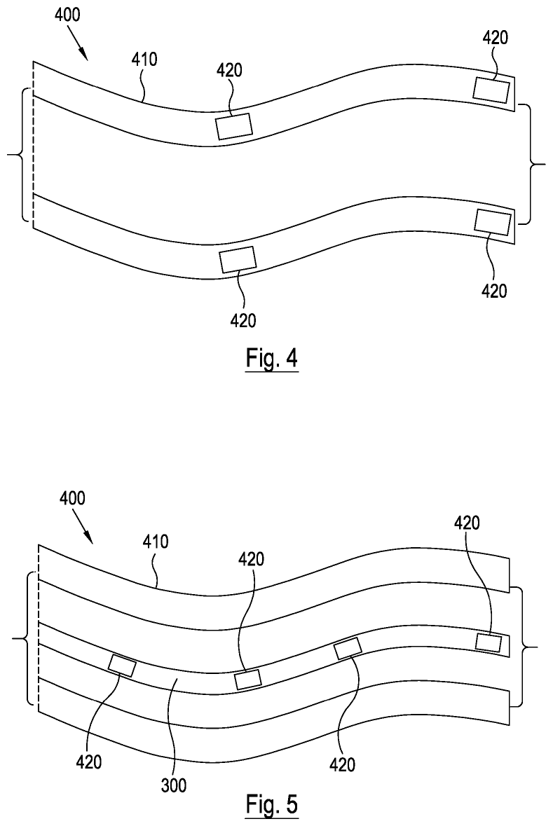

FIGS. 4 and 5 show alternative embodiments of a tube 400 in accordance with the present invention. FIG. 4 corresponds to the embodiment of FIG. 2 and is an axial cross-section of tube 400 having a tube wall 410 and location markers 420 thereon. FIG. 5 corresponds to the FIG. 3 embodiment and shows a tube 400 having a tube wall 410 and a plurality of location markers 420 on a guide wire 300.

Instead of piezo-electric emitters, the passive location markers 420 of the FIGS. 4 and 5 embodiments are laser-etched areas of high echogenicity. An ultrasound wave is applied to the intracorporal area of interest from an emitter source (not shown) located external to the patient's body. When the tube 400 is at least partially inside the intracorporal area of interest, the location markers reflect the externally originating ultrasound signal an externally ultrasound detector/probe (not shown). Each location marker of high echogenicity can be made in such a way that it reflects or modifies the externally applied ultrasound signal differently from the others on the tube 400 and thus each location marker is distinguishable from all other location markers. A further benefit of using a guidewire 300 is that the guidewire itself has a higher echogenicity compared to the rest of the tube 400.

In another embodiment of the present invention (not shown), the location markers 420 of high echogenicity are combined with the emitter-type location markers 120 in a tube which therefore uses both passive and active location markers.

As previously stated, the example of intracorporal tube in FIGS. 2 to 5 is that of a nasogastric tube. However, the intracorporal tube may be another type of tube used in an invasive, blind ended procedure. For example, the tube may be an abdomen draining tube, percutaneous endoscopic gastrostomy (PEG) tube, radiological inserted gastrostomy (RIG) tube, or a catheter, to name a few non-limiting examples.

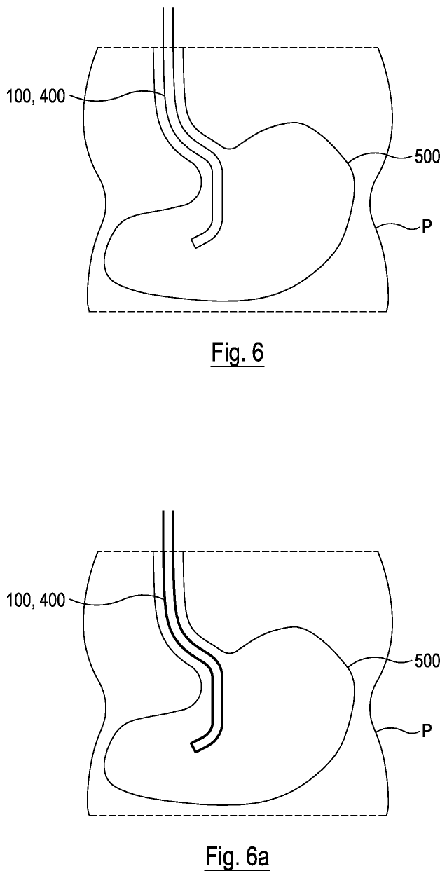

FIG. 6 shows schematically an intracorporal tube 100, 400 within an intracorporal area of interest 500 of a human patient P. In the example of FIG. 6, the tube 100, 400 comprises nasogastric tube and the intracorporal area of interest 500 is a stomach of the patient P.

FIG. 6a shows a schematic example of an ultrasound image of an intracorporal tube 100 comprising a nasogastric tube within a patient's stomach 500. The tube 100 is equipped with a plurality of active markers as described above. When the markers are active, and thus, emitting an ultrasound signal, the nasogastric tube is strongly highlighted on an ultrasound image detected by an ultrasound detector (not shown).

Being able to accurately observe the location of the tube within the patient's stomach facilitates the correct placement of the tube and thereby improves the safety of the procedure.

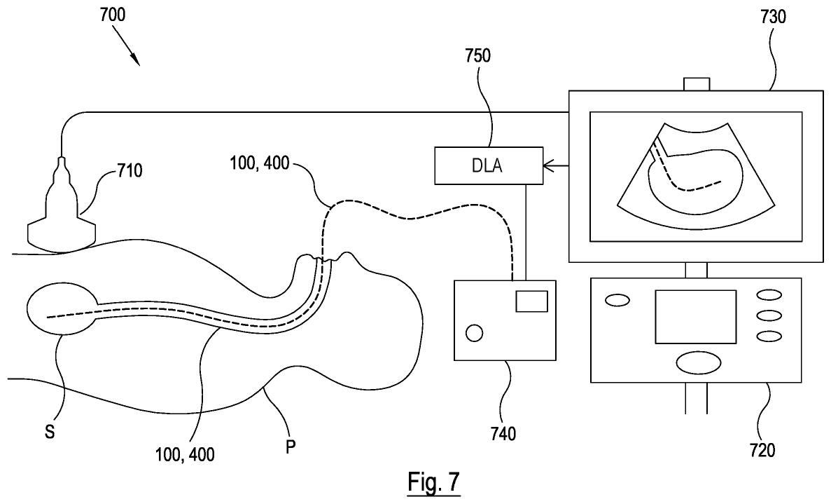

FIG. 7 shows an apparatus 700 for improved detection/positioning of an intracorporal tube 100,400 within the body of a patient P. The apparatus comprises the intracorporal tube either 100 or 400, an ultrasound probe 710 and an imaging signal processing module 720. The apparatus 700 further includes a display monitor 730 for displaying the ultrasound image. When the tube used in apparatus 700 is a tube 100 according to the examples shown in FIGS. 2 and 3, the apparatus further comprises a driver 740 for driving the piezo markers in the tube 100. The ultrasound probe 710 comprises an ultrasound transducer which generates and receives ultrasound signals. The generated ultrasound signal is applied to the intracorporal area of interest, in this case a stomach S of patient P. The ultrasound signal interacts with matter within the region of interest and is reflected back to the probe 710. The reflected ultrasound signal corresponds to anatomical features of the patient, in this case, to dimensions of the patient's stomach and objects within the patient's stomach. The reflected ultrasound waves are received and interpreted by the ultrasound signal processing module 720 to produce a real-time ultrasound image which is displayed on the display 730.

Optionally, a deep learning analysis (DLA) module 750 receives an input from the display 730. The DLA module 750 interacts with the ultrasound image display 730 to localise anatomical regions of interest, such as the stomach, using known techniques (e.g., semantic segmentation). Displaying such identified regions of interest on the live ultrasound image may also help the user find these regions while scanning the patient.

By analysing the area containing the region of interest on the ultrasound image, the DLA module 750 can determine whether any of the location markers are within that region. As an example, this method could be used to determine whether the marker representing the end of the NG tube is inside the stomach.

Where apparatus 700 uses a tube 400, including location markers made from areas of high echogenicity, the ultrasound probe 710 receives a stronger reflected signal from the reflective elements on the tube, as compared to the surrounding area, causing the tube 400 to stand out against the surrounding imaged area on the real time ultrasound image presented on the display 730.

Where apparatus 700 uses a tube 100 including location markers which are themselves ultrasound emitters, the ultrasound probe 710 receives ultrasound waves emitted from the ultrasound-emitting location markers, making the tube much more visible on the ultrasound image against the surrounding imaged area.

Where the active markers can support it, a Doppler imaging technique is enabled by using multiple sources of ultrasound emission. Distance information can be obtained by analysing the relative phase changes between received ultrasound waves from the multiple sources of ultrasound emission. Doppler imaging of the received ultrasound waves may provide an even more detailed image of the tube 100, 400 within the patient's stomach.

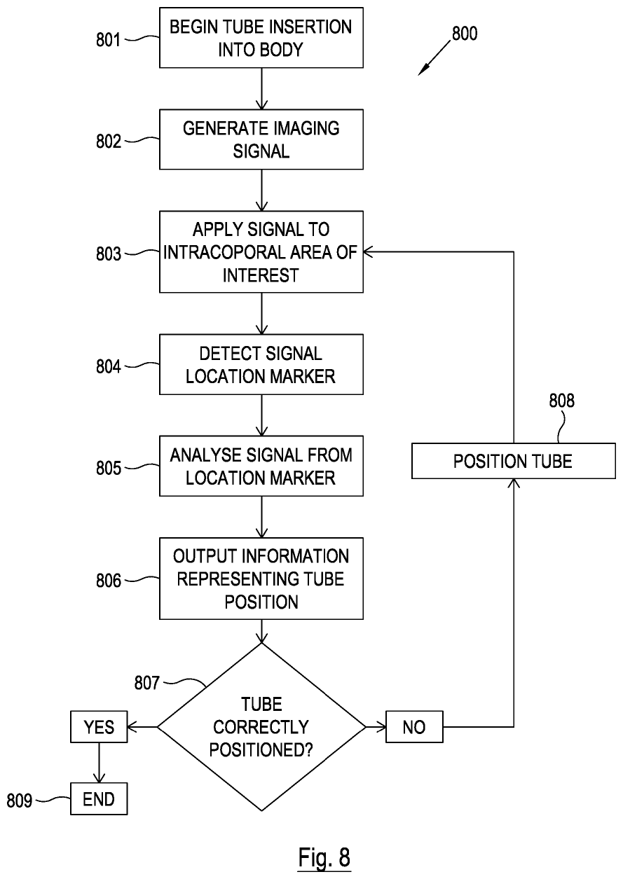

FIG. 8 shows schematically a method 800 for improved detection/positioning of an intracorporal tube, comprising at least one location marker, in a body. At 801, inserting the tube into a body of a human or animal is begun. At 802, an imaging signal is generated. At 803, the image signal is applied to the intracorporal area of interest. At 804, an imaging signal is detected from the at least one location marker. At 805, the imaging signal from the at least one location marker is analysed. At 806, information representing a position of the tube within the intracorporal area of interest is output. At 807, it is queried whether the tube is correctly positioned. If the tube is correctly positioned, this part of the method may end 809. If the tube is not correctly positioned, the method continues with step 808, wherein the position of the tube is changed whilst referring to the information representing a position of the tube. Steps 804 to 807 are then repeated. Steps 804 to 808 are repeated cyclically until it is established that the tube is in the correct position. Steps 804 to 808 take place contemporaneously and continuously to obtain a real time image of the tube within the area of interest.

Where the tube comprises a guidewire, the method further comprises the step of removing the guidewire from the body after correct positioning of the tube. The output information can be transmitted to a signal processing module to produce a real-time image of the tube within the intracorporal area of interest which can then be displayed on a display for a user of the apparatus to view.

The method 800 of FIG. 8 will be further explained in relation to carrying out a nasogastric tube feeding procedure and using ultrasound waves as the imaging signal. At 801, the tube enters the body through the nose. At 802, an ultrasound signal is generated and, at 803, said ultrasound signal is applied to the oesophagus and/or stomach. At 804, an ultrasound signal is detected from the at least one location marker. At 805, the ultrasound signal from the at least one location marker is analysed. At 806, information representing a position of the tube within the oesophagus and/or stomach is output. At 807, it is queried whether the nasogastric tube is correctly positioned. If the nasogastric tube is correctly positioned, the method may end 809. If the nasogastric tube is not correctly positioned, the method continues with step 808, wherein the position of the tube is altered within the oesophagus or stomach. Steps 804 to 807 are then repeated. Steps 804 to 808 are repeated cyclically until it is established that the tube is in the correct position. Steps 804 to 808 take place contemporaneously and continuously to obtain a real time image of the tube within the oesophagus or stomach. Where the method 800 uses the tube 400, wherein the at least one location marker is an ultrasound reflective element, at 804, the ultrasound signal detected from the ultrasound reflective element is an ultrasound signal originating from an ultrasound probe reflected from the ultrasound reflective element. Where the method 800 uses the tube 100, wherein the at least one location marker is an ultrasound emitter, at 804, the ultrasound signal detected from the ultrasound emitter is an ultrasound signal originating directly from the ultrasound emitter itself. In an example of method 800, there is a plurality of location markers in the form piezo-electric transducers. In this example, the method further comprises vibrating each of the piezo-electric emitters at different frequencies. Furthermore, the method may further comprise pulsating the vibrations of each of the piezo-electric transducers. This allows differentiation between each of the plurality of location markers.

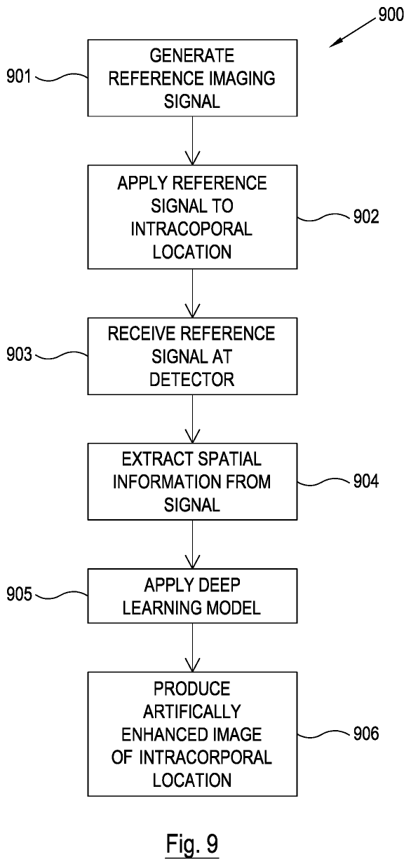

FIG. 9 shows a method 900 for producing an artificially enhanced image of the intracorporal area of interest wherein an intracorporal tube is to be positioned according to method 800. Method 900 can be performed before, or contemporaneously with, method 800. At 901, a reference image signal is generated and, at 902, applied to the intracorporal area of interest. At 903, the reference imaging signal is received from the intracorporal area of interest at a detector. At 904, spatial information from the detected imaging signal is extracted. At 905, deep learning modelling techniques are applied to the extracted information to identify and locate the intracorporal area of interest, and to produce an artificially enhanced image of the intracorporal area of interest 906. As the system knows both the location of the intracorporal area of interest and the position of the location markers on the intracorporal tube, it can determine which parts of the tube (if any) lie within the intracorporal area of interest.

The method 900 of FIG. 9 will be further explained in relation to carrying out a nasogastric tube feeding procedure and using ultrasound waves as the imaging signal. Method 900 produces an artificially enhanced image of a patient's stomach wherein a nasogastric tube is to be positioned. At 901, an ultrasound transducer generates a reference ultrasound signal which is applied, at 902, to the stomach. At 903, the reference ultrasound signal is received from the stomach back at the ultrasound transducer and an ultrasound signal processing module uses the received reference ultrasound signal to produce a real-time image. At 904, the real-time image is transmitted to a real-time segmentation unit wherein the anatomical information in the ultrasound image is interpreted. At 905, the real-time segmentation unit applies standard deep learning modelling techniques to the real-time image to locate, identify and classify the imaged stomach. Predicted spatial parameters of the patient's stomach are overlaid onto the real-time ultrasound image 906. Further information retrieved from the deep learning model can be overlaid onto the real-time ultrasound image shown on a display. The application of deep learning models to the real-time ultrasound image of a patient's stomach in combination with imaging of the tube produces an artificially enhanced image of the tube and the patient's stomach. As the system can determine the position of the NG tube from the location markers, and can locate the stomach on the ultrasound image, it is able to determine which parts of the tube (if any) lie within the stomach.

This allows a technically challenging procedure, such as the nasogastric tube feeding procedure, to be carried out more safely. Since method 900 applied to method 800 provides a real-time, clear image of an intracorporal tube in an intracorporal area with additional useful information and instruction, technical intracorporal procedures, such as nasogastric tube feeding procedure, need not be carried out by an experienced clinician. Using the above-described methods 800, 900, intracorporal tube insertion procedures may be carried out by a non-specialised operative. This may alleviate staffing pressure, for example, at a hospital. It may also alleviate procedure waiting time, since it is no longer necessary to wait for particular personnel to become available.

Apparatus and methods in accordance with the present invention provide several benefits. In addition to allowing the confirmation of placement of the tube, the location and its relationship with the patient's anatomy can be checked at any time, for example when there is a risk of the tube becoming displaced. This may happen when the patient becomes agitated or confused. In addition, when the tube is in place for longer periods, its position may be checked prior to every feed.

The active location markers can interact with the ultrasound scanning apparatus, for example such that the scanner makes one of the markers emit a signal and monitors for the corresponding signal on the ultrasound image. This can be combined with an artificial intelligence/deep learning model that localises the region of interest on the viewed ultrasound image, thereby allowing a highly accurate determination of the placement of the marker, and hence the specific part of the tube.

As a result, embodiments of the present invention provide better patient safety, in that there is a reduction in errors of tube placement, a reduction in a delay to commencement of feeding and a reduction in “hand offs” of care, for example contacting clinicians to review X-rays for patients with whom they have not previously been involved.

The patient's experience may be improved, as he or she is not required to leave the ward and may begin feeding sooner.

Staff time, and therefore costs, are reduced as doctors need not be involved in requesting x-rays and nurses do not need to organise the transfer of patients to the radiology department. Furthermore, the costs of porters, radiographers and radiologists need not be incurred.

Staff efficiency is improved by allowing more junior staff to undertake confirmation of tube position, thereby allowing more senior staff to undertake more value-added tasks.

The location markers may be placed on the outside of the tube, embedded within the tube wall itself or else on the removable guide wire down the middle of the tube.

Although the examples given above are of a gastrointestinal tube, embodiments of the present invention may have other medical uses, such as (but not limited to): surgical drains, chest drains (pleural/thoracic or cardiac), percutaneous nephrostomy, and bile duct drainage.

Whilst endeavouring in the foregoing specification to draw attention to those features of the invention believed to be of particular importance, it should be understood that the applicant claims protection in respect of any patentable feature or combination of features referred to herein, and/or shown in the drawings, whether or not particular emphasis has been placed thereon.

1. An intracorporal tube for positioning within a patient's body, the tube comprising at least one location marker on, in, or within the tube, the presence and/or position of which marker is detectable by an ultrasound imaging system.

2. An intracorporal tube according to claim 1, wherein the tube has a plurality of location markers at different locations on, or within, the tube.

3. An intracorporal tube according to claim 1, wherein the tube has one or more active location markers and/or one or more passive location markers.

4. An intracorporal tube according to claim 1, wherein the at least one location marker is provided in or on a wall portion of the tube.

5. An intracorporal tube according to claim 1, wherein the at least one location marker is provided in or on a guide wire of the tube.

6. An intracorporal tube according to claim 1, wherein, where there is a plurality of location markers, at least two are configured to produce differentiable imaging signals in an imaging system such that the positions of the location markers are distinguishable.

7. An intracorporal tube according to claim 1, wherein at least one location marker is arranged in use to generate, reflect, or modify an ultrasound wave as the imaging signal.

8. An intracorporal tube according to claim 1, wherein at least one location marker is a passive marker and comprises a region of high echogenicity.

9. An intracorporal tube according to claim 1, wherein one or more of the location markers comprises both an active marker and a passive marker.

10. An intracorporal tube according to claim 1, wherein active location markers are arranged in use to vibrate at different frequencies to produce differentiable ultrasound signals.

11. An intracorporal tube according to claim 1, wherein active location markers are configured to pulse or vibrate in an identifiable or distinguishable pattern which may be used to distinguish between them.

12. An intracorporal tube according to claim 1, wherein the tube comprises a gastric tube for use in a nasogastric or orogastric tube feeding or aspiration procedure.

13. An intracorporal tube according to claim 1, wherein the at least one location marker comprises an etched portion of the tube, such as a laser-etched portion.

14. An apparatus for improved detection and/or positioning of an intracorporal tubing within a body, the apparatus comprising: an intracorporal tube; an ultrasound imaging signal detector; and an ultrasound imaging signal processing module.

15. An apparatus according to claim 14, comprising an ultrasound imaging signal source.

16. An apparatus according to claim 14, wherein the intracorporal tube is for positioning within a patient's body, the tube comprising at least one location marker on, in, or within the tube, the presence and/or position of which marker is detectable by an ultrasound imaging system.

17. A method for improved detection and/or positioning of an intracorporal tube in a patient's body, the method comprising:
inserting the intracorporal tube into the patient's body, the intracorporal tube comprising at least one location marker; detecting an ultrasound imaging signal from the at least one location marker; and using said ultrasound imaging signal to determine the presence of the or each location marker within the patient's body and/or their spatial relationship.

18. A method according to claim 17, wherein the method further comprises processing information representing a position of the or each location marker tube within the patient's body to produce a real-time image of the tube and/or its location.

19. A method according to claim 17, wherein the method further comprises displaying the real-time image of the tube in the patient's body on a display/monitor.

20. A method according to claim 17, wherein the method further comprises extracting spatial information from the detected imaging signal and applying a deep learning model to the extracted information to produce an artificially enhanced image of an intracorporal area of interest.

21. A method according to claim 17, wherein when the, or each, location marker comprises an active marker the method comprises using Doppler imaging to interpret imaging signals received from the location marker for improved detection and/or positioning of the tube within the patient's body.

22. A method according to claim 17, wherein when the location markers are located on a guidewire of the tube, the method comprises removing the guidewire from the body after correct positioning of the tube.

23. A method according to claim 17, wherein, where there is a plurality of location markers the method comprises causing the location markers to vibrate at different frequencies and/or to pulse the ultrasound wave of the location markers at a different frequency or in a different pattern, to differentiate between the plurality of location markers.

24. A method according to claim 20, comprising ascertaining the location of the intracorporal area of interest and the position of the location markers on the intracorporal tube, and determining which parts of the tube (if any) lie within the intracorporal area of interest.
